(19) United States
(12) Patent Application Publication
(10) Pub. No.: US 2013/0149750 A1
(43) Pub. Date: Jun. 13, 2013

(54) METHOD FOR PRETREATMENT OF CELLULOSE FOR FERMENTATION IN AN AQUEOUS ENVIRONMENT

(71) Applicant: Quad County Corn Processors, Galva, IA (US)

(72) Inventor: Travis Brotherson, Holstein, IA (US)

(73) Assignee: Quad County Corn Processors, Galva, IA (US)

(21) Appl. No.: 13/706,028

(22) Filed: Dec. 5, 2012

Related U.S. Application Data
(60) Provisional application No. 61/567,798, filed on Dec. 7, 2011.

(51) Int. Cl. C12P 19/14

(52) U.S. Cl. CPC C12P 19/14
USPC 435/99

(57) ABSTRACT

A method for pretreating cellulose for fermentation in an aqueous environment comprises the steps of: grinding a biomass to reduce its particle size, adding water to the ground biomass to create a slurry, soaking the slurry, heating the slurry at a first pressure greater than atmospheric pressure, reducing the pressure of the slurry, reducing the temperature of the slurry, determining whether the enzymes used require preliminary enzymatic hydrolysis, and performing preliminary enzymatic hydrolysis on the slurry if the enzymes used require preliminary enzymatic hydrolysis.

### RELATED APPLICATIONS

The present patent application is a non-provisional utility application claiming priority benefit to earlier-filed provisional application U.S. Application Ser. No. 61/567,798 entitled “METHOD FOR PRETREATMENT OF CELLULOSE FOR FERMENTATION IN AN AQUEOUS ENVIRONMENT”, filed Dec. 7, 2011, and incorporated by reference herein in its entirety.

### BACKGROUND OF THE INVENTION

1. Field of the Invention

Embodiments of the current invention relate to methods for converting cellulosic biomass into carbohydrate chains that are more readily accessible for enzymatic hydrolysis into fermentable sugars.

2. Description of the Related Art

A biomass is a quantity of biological material, typically taken from living or recently living organisms, that is utilized to generate energy. The biomass may include or be derived from plants and trees such as miscanthus, switchgrass, hemp, corn, sorghum, sugarcane, bamboo, poplar, willow, eucalyptus, oil palm, and the like. Among other applications, the biomass may be utilized in the production of ethanol for use as a gasoline additive or a straight liquid fuel. During the production process, cellulose contained in the biomass is generally broken down into carbohydrates that are digested by fermentation organisms. However, the cellulose may be difficult to reduce to carbohydrates that are digestible by currently available fermentation organisms.

### SUMMARY OF THE INVENTION

Embodiments of the current invention solve the above-mentioned problems and provide a distinct advance in the art of methods for preparing a cellulosic biomass for fermentation.

Various embodiments of the current invention include a method for pretreating cellulose for fermentation in an aqueous environment comprising the steps of: grinding a biomass to reduce its particle size, adding water to the ground biomass to create a slurry, soaking the slurry, heating the slurry at a first pressure greater than atmospheric pressure, reducing the pressure of the slurry, reducing the temperature of the slurry, determining whether the enzymes used require preliminary enzymatic hydrolysis, and performing preliminary enzymatic hydrolysis on the slurry if the enzymes used require preliminary enzymatic hydrolysis.

Additional steps of the method include performing fermentation at a temperature lower than the temperature of the preliminary enzymatic hydrolysis on the slurry if the enzymes used require preliminary enzymatic hydrolysis and performing combined enzymatic hydrolysis and fermentation on the slurry if the enzymes used do not require preliminary enzymatic hydrolysis.

Other steps of the method include determining the particle size of the slurry and regrinding the slurry if the particle size is above a first value, determining a water absorption of the slurry and resoaking the slurry if the water absorption is below a second value, and determining an amount of cellulose hydrolyzation of the slurry and reheating the slurry if the amount of cellulose hydrolyzation is below a third value.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the current invention will be apparent from the following detailed description of the embodiments and the accompanying drawing figures.

### BRIEF DESCRIPTION OF THE DRAWING FIGURES

Embodiments of the current invention are described in detail below with reference to the attached drawing figures, wherein:

FIG. 1 is a schematic block diagram of a system, constructed in accordance with various embodiments of the current invention, for pretreating cellulose for fermentation in an aqueous environment; and

FIG. 2 is a flow diagram depicting the steps of a method for pretreating cellulose for fermentation in an aqueous environment.

The drawing figures do not limit the current invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the current invention. The following detailed description is, therefore, not to be taken in a limiting sense. The scope of the current invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current technology can include a variety of combinations and/or integrations of the embodiments described herein.

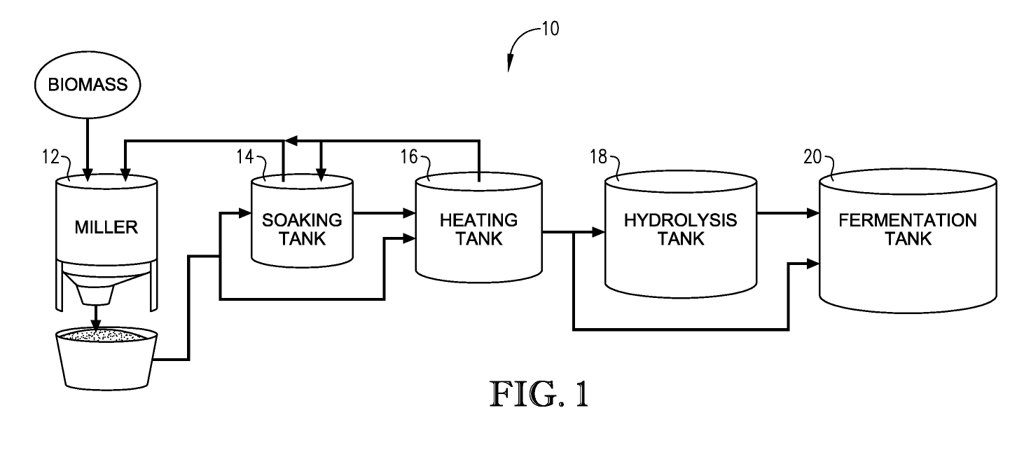

A system 10 for pretreating cellulose for fermentation in an aqueous environment, constructed in accordance with various embodiments of the current invention, is shown in FIG. 1. Cellulose may naturally be present in a biomass that is processed to produce ethanol or related fuels. The biomass may include or be derived from plants and trees such as miscanthus, switchgrass, hemp, corn, sorghum, sugarcane, bamboo, poplar, willow, eucalyptus, oil palm, and the like. The biomass may also include preprocessed products such as sugar cane bagasse, paper mill pulp, whole stillage, and the like. As part of an ethanol production process, cellulose contained in the biomass is broken down into carbohydrates to be digested by organisms during fermentation. Often, the cellulose does not break down sufficiently and the biomass requires pretreatment in order to ferment properly.

The system 10 may broadly comprise a mill 12, a soaking tank 14, a heating tank 16, a hydrolysis tank 18, and a fermentation tank 20. The system 10 may further include biomass processing equipment and components, such as pumps or other transport equipment, that are not shown or discussed herein. The system 10 may be utilized to perform a method 100 for pretreating cellulose for fermentation in an aqueous environment, discussed in more detail below.

The mill 12 may include milling or grinding machines for dry milling, such as a hammer mill, a knife mill, or the like, or for wet milling, such as a high shear mixer, a colloid mill, a conical mill, or the like. The mill 12 is utilized to grind the biomass at various times during the pretreatment method 100 and may include one or more dry mills, one or more wet mills, or combinations thereof.

The soaking tank 14, the heating tank 16, the hydrolysis tank 18, and the fermentation tank 20 may include large volume tanks as are known in the art. The tanks 14, 16, 18, 20 may also be open or sealed, may include the ability to maintain an internal pressure, and may include a heat source. Some tanks 14, 16, 18, 20 may include a direct steam injector or a heat exchanger. The tanks 14, 16, 18, 20 may be named for a function or a portion of the method 100, although more than one function or portion of the method 100 may be performed in each tank 14, 16, 18, 20. Furthermore, the system 10 may include a plurality of each tank 14, 16, 18, 20.

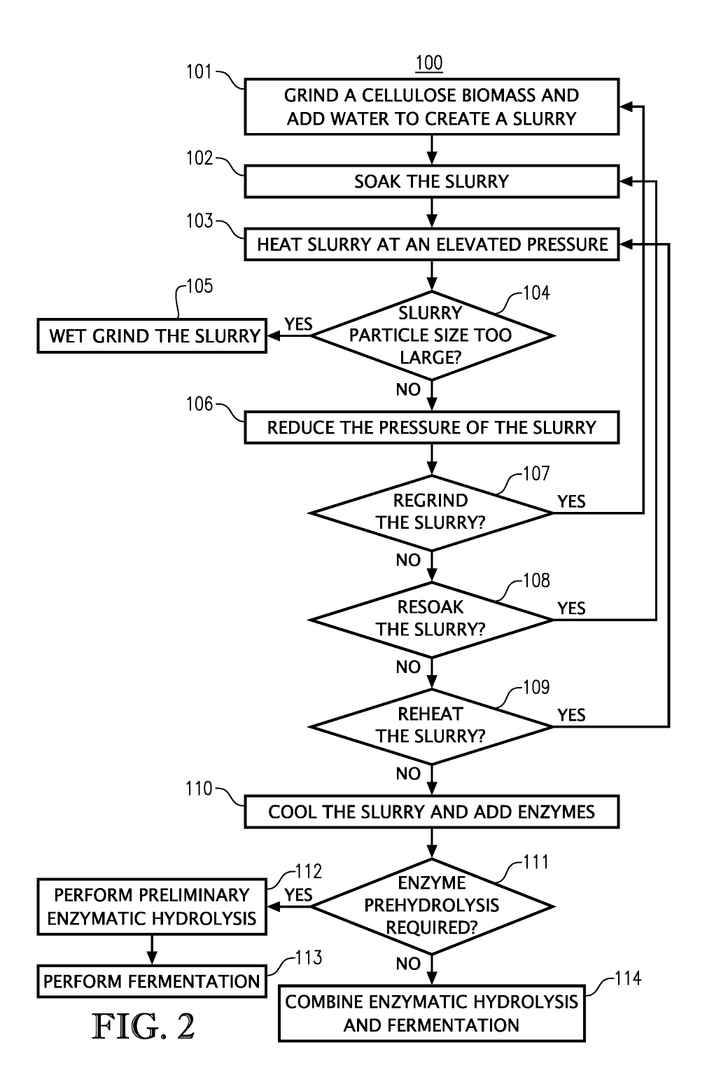

The method 100, in accordance with various embodiments of the current invention, for pretreating the biomass may include the steps shown in FIG. 2. The steps may be performed in the order shown or in a different order. Some steps may be performed concurrently. Furthermore, the method 100 may omit some of the steps shown or may include steps that are not shown.

In step 101, the biomass is ground with the mill 12. Grinding is initially performed to increase the surface area to volume ratio of the biomass by reducing its particle size. The biomass may be ground while it is dry with a dry mill. Alternatively, water, or other liquids, may be added and the combination of biomass and water may be ground with a wet mill. If water is not added before the first grinding of the biomass, then water is added to the biomass after it is ground. Some types of biomass, such as sugar cane bagasse or paper mill pulp, may enter the system 10 in a wet state and may be ground in a wet mill, although water may be added as well. The combination of the biomass and the water may be known as a “slurry”. The water may be fresh, recycled from other steps of the current method 100, recycled from another process, or combinations thereof. The water added may include other solids and does not have to be pure, as long as the contaminants do not interfere with subsequent steps of the method 100.

The amount of water added during this step may be determined by viscosity of the slurry and the ability of the system 10 to handle varying levels of viscosity. For example, if less water is added to the biomass, then viscosity of the slurry is greater, but the slurry may be more difficult to pump between tanks 14, 16, 18, 20 or other components of the system 10—which is less desirable. Typically, enough water is added to maintain a minimal viscosity for pumping.

In step 102, the slurry is soaked to soften it and help break down the cellulose by allowing water molecules to infiltrate the cellulose matrix. Some types of biomass, such as paper mill pulp, may enter the system 10 as a slurry that is already ground and wet, and thus may bypass step 101. The soak time may depend in part on the size of the particles of the slurry. Typically, larger particles require a longer soak time while smaller particles require a shorter soak time, although longer soak times generally increase the breakdown of the cellulose for any size particle. The temperature of the slurry may also affect the soak time. An elevated temperature of the slurry may increase or enhance the breakdown of cellulose, thereby requiring a decreased soak time. Accordingly, a lower temperature may lead to longer soak times.

In various embodiments, one or more acids may be added to the slurry while it is soaking to enhance cellulose breakdown. The polar nature of the acid allows electromagnetic bonding to areas of the cellulose, which subsequently become detached. This aids in the infiltration of water molecules into the cellulose matrix.

In various embodiments, the slurry may be recycled through the mill 12 to provide continual grinding which helps to break down the cellulose.

In step 103, the slurry is heated under pressure. The slurry may be transferred from the soaking tank 14 to the heating tank 16 so that it can be heated at a pressure greater than atmospheric pressure. Some types of biomass, such as whole stillage of paper mill pulp, may enter the system 10 as a slurry that is already ground and soaked, and thus may bypass steps 101 and 102. In some embodiments, the heating may occur by directly injecting steam onto or into the slurry. Cavitation may occur. In other embodiments, a heat exchanger or other heat source component may be used to heat the slurry. The temperature may be raised to a range from approximately 215° Fahrenheit (F.) to approximately 350° F. The specific temperature may depend in part on the type of biomass used, the extent to which the biomass or slurry was ground, and the time period that the slurry was soaked.

Step 104 determines whether the slurry particle size is too large. Larger slurry particles may require increased soak times and higher temperatures to break down the cellulose. If the particle size of the slurry is too large, then the method 100 transfers to step 105. If the slurry does not need any more grinding, then the method transfers to step 106.

In step 105, the slurry is ground again. The slurry may be ground with a wet mill at elevated temperature and pressure. Then, the slurry may be transferred back to the heating tank 16 or the slurry may remain in the mill 12.

In step 106, the pressure of the heating tank 16 or the mill 12 is reduced rapidly. The reduction of pressure may be accomplished by flashing steam off of the slurry. The steam can either be vented to the atmosphere or directed to a cooling condenser. As a result of the reduced pressure, the slurry is brought to a rapid boil, which expands the water contained in the cellulose, causing a rupturing of the cellulose. The particle size of the slurry may also be reduced.

In steps 107, 108, and 109, the particle size of the slurry may again be evaluated. If the particle size is still above a certain threshold, then the slurry may be ground again and the method 100 transfers to step 101. Otherwise, the water absorption level of the slurry is determined. If the amount of water absorbed into the cellulose matrix is below a certain level, the method transfers to step 102, and the slurry may be resoaked in order to increase water absorption into the cellulose matrix. Or, the amount of cellulose hydrolyzation is determined. If the cellulose hydrolyzation is below a certain level, the method 100 transfers to step 103, and the slurry is reheated to increase cellulose rupturing.

In step 110, the slurry is cooled and enzymes are added. Enzymes may convert the cellulose into usable sugars. In addition, the pH level of the slurry may be adjusted to a level conducive to the activity of the enzymes. In some embodiments, ammonia or chemicals may be added to increase the pH level.

In step 111, the type of enzyme added is determined. Some enzymes require a higher temperature to be active than is used for fermentation. If the enzyme used requires a higher temperature, then a prehydrolysis step may be necessary and the method 100 transfers to step 112. If not, then the method 100 transfers to step 114. In addition, the viscosity of the slurry may be considered. If the viscosity of the slurry is still high, then the slurry may be kept at a higher temperature while the enzymes are added. In this case, the method 100 also transfers to step 112.

In step 112, the slurry undergoes preliminary enzymatic hydrolysis. The slurry may be transferred to the hydrolysis tank 18 and held at a higher temperature while the enzymes are added and continue to release sugars from the cellulose.

In step 113, the slurry is fermented. The temperature of the hydrolysis tank 18 may be reduced, or the slurry may be transferred to the fermentation tank 20 held at a lower temperature.

In step 114, the slurry undergoes enzymatic hydrolysis and fermentation. The slurry may be transferred to the fermentation tank 20 and held at a fermentation temperature. The combination of hydrolysis and fermentation may occur as described in steps 112 and 113.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A method for pretreating cellulose for fermentation in an aqueous environment, the method comprising the steps of:
a) grinding a biomass to reduce its particle size;
b) adding liquid to the ground biomass to create a slurry;
c) soaking the slurry;
d) heating the slurry at a first pressure greater than atmospheric pressure;
e) reducing the pressure of the slurry;
f) reducing the temperature of the slurry;
g) adding enzymes to the slurry;
h) determining whether the enzymes used require preliminary enzymatic hydrolysis; and
i) performing preliminary enzymatic hydrolysis on the slurry if the enzymes used require preliminary enzymatic hydrolysis.

2. The method of claim 1, further comprising the step performed after step i) of performing fermentation at a temperature lower than the temperature of the preliminary enzymatic hydrolysis on the slurry if the enzymes used require preliminary enzymatic hydrolysis.

3. The method of claim 1, further comprising the step performed after step i) of performing combined enzymatic hydrolysis and fermentation on the slurry if the enzymes used do not require preliminary enzymatic hydrolysis.

4. The method of claim 1, further comprising the step performed after step d) of determining the particle size of the slurry and regrinding the slurry at the first pressure if the particle size is above a first value.

5. The method of claim 1, further comprising the step performed after step e) of determining the particle size of the slurry and regrinding the slurry if the particle size is above a first value.

6. The method of claim 1, further comprising the step performed after step e) of determining a water absorption of the slurry and resoaking the slurry if the water absorption is below a first value.

7. The method of claim 1, further comprising the step performed after step e) of determining an amount of cellulose hydrolyzation of the slurry and reheating the slurry if the amount of cellulose hydrolyzation is below a first value.

8. A method for pretreating cellulose for fermentation in an aqueous environment, the method comprising the steps of:
a) grinding a biomass to reduce its particle size;
b) adding liquid to the ground biomass to create a slurry;
c) soaking the slurry;
d) heating the slurry at a first pressure greater than atmospheric pressure;
e) reducing the pressure of the slurry;
f) reducing the temperature of the slurry;
g) adding enzymes to the slurry;
h) determining whether the enzymes used require preliminary enzymatic hydrolysis;
i) performing preliminary enzymatic hydrolysis on the slurry if the enzymes used require preliminary enzymatic hydrolysis;
j) performing fermentation at a temperature lower than the temperature of the preliminary enzymatic hydrolysis on the slurry if the enzymes used require preliminary enzymatic hydrolysis; and
k) performing combined enzymatic hydrolysis and fermentation on the slurry if the enzymes used do not require preliminary enzymatic hydrolysis.

9. The method of claim 8, further comprising the step performed after step d) of determining the particle size of the slurry and regrinding the slurry at the first pressure if the particle size is above a first value.

10. The method of claim 8, further comprising the step performed after step e) of determining the particle size of the slurry and regrinding the slurry if the particle size is above a first value.

11. The method of claim 8, further comprising the step performed after step e) of determining a water absorption of the slurry and resoaking the slurry if the water absorption is below a first value.

12. The method of claim 8, further comprising the step performed after step e) of determining an amount of cellulose hydrolyzation of the slurry and reheating the slurry if the amount of cellulose hydrolyzation is below a first value.

13. A method for pretreating cellulose for fermentation in an aqueous environment, the method comprising the steps of:
a) grinding a biomass to reduce its particle size;
b) adding liquid to the ground biomass to create a slurry;
c) soaking the slurry;
d) heating the slurry at a first pressure greater than atmospheric pressure;
e) determining the particle size of the slurry and regrinding the slurry at the first pressure if the particle size is above a first value;
f) reducing the pressure of the slurry;
g) determining the particle size of the slurry and regrinding the slurry if the particle size is above the first value;
h) determining a water absorption of the slurry and resoaking the slurry if the water absorption is below a second value;
i) determining an amount of cellulose hydrolyzation of the slurry and reheating the slurry if the amount of cellulose hydrolyzation is below a third value;
j) reducing the temperature of the slurry;
k) adding enzymes to the slurry;
l) determining whether the enzymes used require preliminary enzymatic hydrolysis;
m) performing preliminary enzymatic hydrolysis on the slurry if the enzymes used require preliminary enzymatic hydrolysis;
n) performing fermentation at a temperature lower than the temperature of the preliminary enzymatic hydrolysis on the slurry if the enzymes used require preliminary enzymatic hydrolysis; and
o) performing combined enzymatic hydrolysis and fermentation on the slurry if the enzymes used do not require preliminary enzymatic hydrolysis.
